(19) United States
(12) Patent Application Publication
(10) Pub. No.: US 2017/0128000 A1
(43) Pub. Date: May 11, 2017

(54) METHOD AND APPARATUS FOR MONITORING A PHYSIOLOGICAL INDICATION ASSOCIATED WITH FUNCTIONING OF A LIVING ANIMAL

(71) Applicant: CAPTURE ANALYTICS INC., Calgary (CA)

(72) Inventors: Steven Ross MARTIN, Calgary (CA); Grant Veralyn LOWE, Calgary (CA)

(21) Appl. No.: 15/321,959

(22) PCT Filed: Jun. 23, 2015
(86) PCT No.: PCT/CA2015/000413

§ 371 Date: Dec. 23, 2016

Related U.S. Application Data
(60) Provisional application No. 62/019,148, filed on Jun. 30, 2014.

(51) Int. Cl. A61B 5/00; A61B 5/11; A61N 7/00; A61B 5/0488

(52) U.S. Cl. CPC A61B 5/4557; A61B 5/0488; A61B 5/1107; A61B 5/6814; A61B 5/4818; A61N 7/00; A61B 2562/0261; A61B 2562/164

(57) ABSTRACT

A method and apparatus for monitoring a living animal is disclosed. The apparatus includes a flexible carrier strip having an undersurface for adhering to an epidermis of the living animal. The apparatus also includes a muscle function sensor disposed on the flexible carrier strip and operable to generate a muscle signal indicative of functioning of a muscle underlying the flexible carrier strip. The apparatus further includes a transducer disposed on the flexible carrier strip operable to generate a stimulus signal in response to receiving mechanical stimuli, a processor circuit disposed on the flexible carrier strip. The processor circuit includes a sensor interface in communication with the muscle function sensor and the transducer for receiving the muscle and stimulus signals, a microprocessor operably configured to process the signals to produce a physiological indication associated with functioning of the living animal, a memory operable to store data representative of variations in the physiological indications over a period of time, and a communications interface for communicating stored data to an output device.

### BACKGROUND OF THE INVENTION

1. Field of Invention

This invention relates generally to monitoring physiological indications associated with functioning of a living animal and more particularly to a monitoring apparatus and method for producing the physiological indications.

2. Description of Related Art

Living animals carry out various physiological functions, including mechanical, physical, bioelectrical, and biochemical functions, that keep the animal alive and functioning. Many of these functions produce observable physiological indications while in progress, such as physical movement of tissues, a temperature increase, and generation of sounds. At a lower level there may be other more subtle physiological changes in tissues such as a change in electrical impedance or the generation of action potentials for initiating functions such as muscle activation.

The physiological indications may be indicative of either normal or abnormal functioning of the living animal. One example of an abnormal condition in humans is Bruxism, which involves grinding of the teeth and/or excessive clenching of the jaw while sleeping.

There remains a need for methods and apparatus for monitoring physiological indications in living animals including humans and other animals.

### SUMMARY OF THE INVENTION

In accordance with one aspect of the invention there is provided an apparatus for monitoring a living animal. The apparatus includes a flexible carrier strip having an undersurface for adhering to an epidermis of the living animal. The apparatus also includes a muscle function sensor disposed on the flexible carrier strip and operable to generate a muscle signal indicative of functioning of a muscle underlying the flexible carrier strip. The apparatus further includes a transducer disposed on the flexible carrier strip operable to generate a stimulus signal in response to receiving mechanical stimuli, a processor circuit disposed on the flexible carrier strip. The processor circuit includes a sensor interface in communication with the muscle function sensor and the transducer for receiving the muscle and stimulus signals, a microprocessor operably configured to process the signals to produce a physiological indication associated with functioning of the living animal, a memory operable to store data representative of variations in the physiological indications over a period of time, and a communications interface for communicating stored data to an output device.

The muscle function sensor may include a pair of electrodes for sensing an electrical potential associated with functioning of the muscle, the electrodes being disposed on the undersurface of the flexible carrier strip.

The muscle function sensor may include at least one of a force sensor disposed to sense a clamping force associated with activation of the muscle, and a strain gauge disposed to sense strain in the epidermis underlying the flexible carrier strip.

The muscle function sensor may be operably configured to produce a muscle signal indicating a force associated with the functioning of the muscle.

The transducer may include a microphone and the mechanical stimuli may include sound waves.

The microphone may be operable to produce a stimulus signal that facilitates determination of a sound pressure level of sound waves incident on the microphone.

The transducer may include a vibration transducer and the mechanical stimuli may include vibration waves.

The transducer may include a motion detector and the mechanical stimuli may include movements of the living animal.

The stimulus signal may be operable to provide an indication of an orientation of a portion of the living body to which the flexible carrier strip is adhered.

The sensor interface may include a signal conditioner for receiving the muscle and stimulus signals and converting the signals into a form suitable for processing by the microprocessor.

The flexible carrier strip may be configured to be adhered to the epidermis of a human for producing physiological indications associated with sleep disorders.

The flexible carrier strip may be configured to be adhered to the epidermis in one of a jaw area and a facial area.

The physiological indications associated with sleep disorders may include at least one of clenching of jaw muscles associated with bruxism, snoring, and sleep apnea.

The communications interface may be operable to generate signals for communication with a playback device for playing back received periodic mechanical stimuli.

The processor circuit may be operably configured to further process the stored data to produce an abridged version of the received periodic mechanical stimuli for playback.

The microprocessor may be operably configured to process the signals by processing the muscle and stimulus signals to identify physiological events in each of the signals, and identifying a time correspondence between physiological events in the respective signals.

The apparatus may include an ultrasonic transducer disposed on the flexible carrier strip and operable to receive an excitation signal for delivering a dose of therapeutic ultrasound radiation to the muscle underlying the flexible carrier strip.

The apparatus may include an ultrasonic transceiver disposed on the flexible carrier strip and the processor circuit may be operably configured to cause the ultrasonic transceiver generate a pulse of ultrasonic radiation for delivery to tissues of the living body underlying the flexible carrier strip, cause the ultrasonic transceiver receive a signal representing reflections of the ultrasonic waveform from the tissues, and process the signal received by the ultrasonic transducer to produce the physiological indication associated with functioning of the living animal.

Other aspects and features of the present invention will become apparent to those ordinarily skilled in the art upon review of the following description of specific embodiments of the invention in conjunction with the accompanying figures.

### BRIEF DESCRIPTION OF THE DRAWINGS

In drawings which illustrate embodiments of the invention,

FIG. 1 is a perspective view of a monitoring apparatus in accordance with a first embodiment of the invention;

FIG. 2 is a perspective view of the monitoring apparatus shown in FIG. 1 on a human subject for monitoring physiological indications;

FIG. 3 is a schematic view of a processor circuit used in implementing the

FIG. 4 is a plan view of an undersurface of the monitoring apparatus shown in FIG. 1;

FIG. 5 is a plan view of an outer surface of the monitoring apparatus shown in FIG. 1;

FIG. 6 is a plan view of an undersurface of a monitoring apparatus in accordance with an alternative embodiment of the invention; and

FIG. 7 is a plan view of an outer surface of the monitoring apparatus shown in FIG. 6.

### DETAILED DESCRIPTION

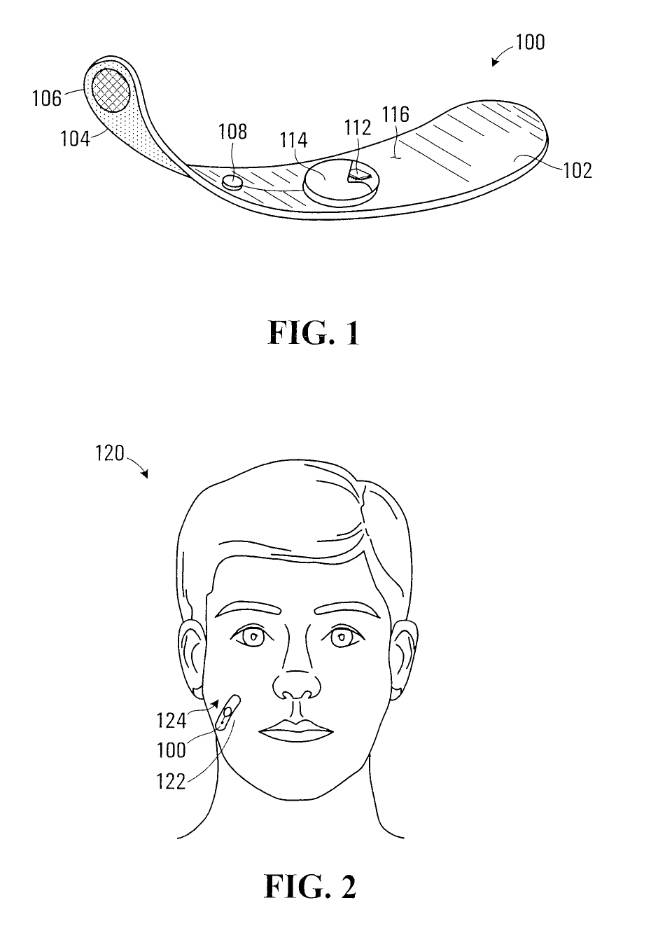

Referring to FIG. 1, a monitoring apparatus for monitoring a living animal according to a first embodiment of the invention is shown generally at 100. The apparatus 100 includes a flexible carrier strip 102 having an undersurface 104, configured for adhering to an epidermis of a living animal. Referring to FIG. 2, in one embodiment the living animal is a human subject 120 and the monitoring apparatus 100 is adhered to an epidermis 122 of the subject. In the embodiment shown the monitoring apparatus 100 is adhered to the epidermis 122 of the human subject 120 proximate the jaw area 124.

Referring back to FIG. 1, the apparatus 100 also includes a muscle function sensor 106 disposed on the flexible carrier strip 102. The muscle function sensor 106 is operable to generate a muscle signal indicative of functioning of a muscle underlying the flexible carrier strip. The monitoring apparatus 100 also includes a transducer 108 disposed on the flexible carrier strip 102, which is operable to generate a stimulus signal in response to receiving mechanical stimuli. Examples of possible mechanical stimuli that may be received include sound waves, vibration waves, and movements associated with the living animal.

The monitoring apparatus 100 further includes a processor circuit 110 disposed on an outer surface 116 of the flexible carrier strip 102. In this embodiment the processor circuit 110 is encapsulated within a housing 114 and includes a microprocessor 112.

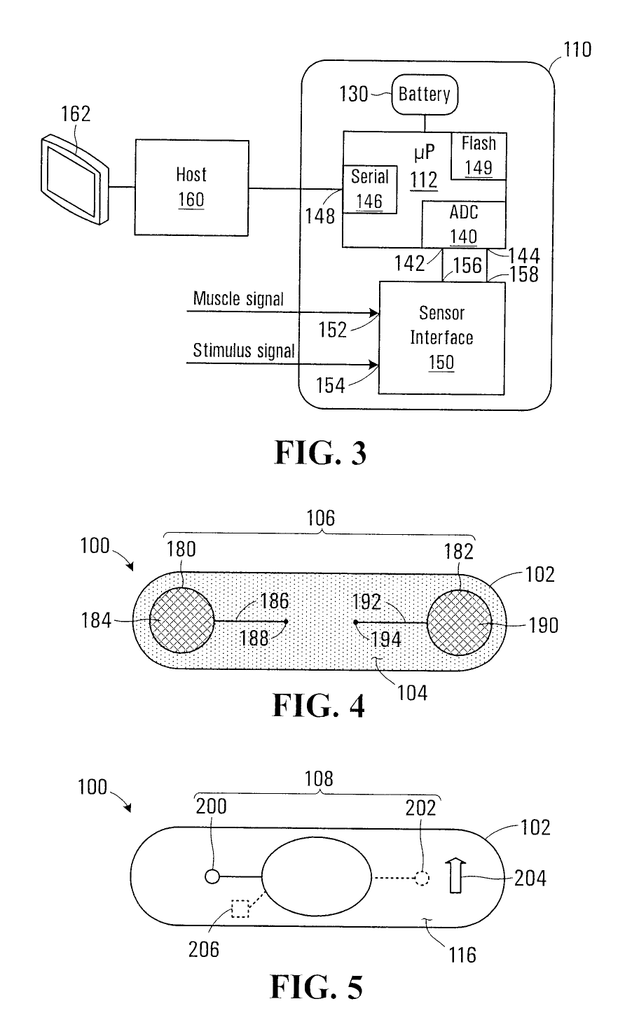

A schematic diagram of one possible embodiment of the processor circuit 110 is shown in FIG. 3. Referring to FIG. 3, the microprocessor 112 is powered by a battery 130 and in one embodiment may be implemented using a low power microcontroller such as the picoPower™ microcontroller produced by Atmel Corporation of San Jose, Calif., USA. The microprocessor 112 further includes an analog to digital signal converter 140, which may include multiple channels, each having a respective input. In FIG. 3, two such inputs 142 and 144 are shown. The microprocessor 112 also includes a communications interface 146, having a port 148 for interfacing with an external host system 160. The communications interface 146 may me implemented as a two-wire serial communications interface, for example. The microprocessor 112 also includes on-board flash memory 149 for storing program instructions and data.

The processor circuit 110 also includes a sensor interface 150 having an input 152 for receiving muscle signals from the muscle function sensor 106 and an input 154 for receiving stimulus signals from the transducer 108. The sensor interface 150 includes signal conditioning circuitry for conditioning the muscle and stimulus signals received at the inputs 152 and 154. The muscle and stimulus signals may typically be received as analog signals and the signal conditioning may involve analog processing such as amplification, rectification, buffering and level shifting, for example. The sensor interface 150 also includes outputs 156 and 158 for connecting to the conditioned signals to the inputs 142 and 144 of the ADC 140. The signal conditioning converts the muscle and stimulus signals into a suitable form for conversion into digital signals by the ADC 140.

The microprocessor 112 receives the conditioned muscle and stimulus signals at the inputs 142 and 144 of the ADC 140, which converts the signals into digital representations for processing by the microprocessor to produce the physiological indication associated with functioning of the living animal. The microprocessor 112 stores data representative of variations in the physiological indications over a period of time in the flash memory 149. In one embodiment, the flash memory 149 is selected to provide sufficient storage for recording about 8 to 9 hours of data for monitoring sleeping patterns of the human subject 120 shown in FIG. 2.

The serial communications interface 146 facilitates connection to the host 160 for communicating the stored data representative of the physiological indications to an output device 162, such as a display monitor. The host 160 and output device 162 may be implemented as a general purpose computer and display, smart-phone, tablet computing device, custom docking station, or any other device operable to receive and display data. In other embodiments communication between the processor circuit 110 and the host 160 may be implemented using a wireless communication protocol such as Bluetooth^{®} or ANT+™ interface, for example. The external host system 160 and output device 162 may be used to play back stored physiological indication data that is generated by the monitoring apparatus 100. The playback may involve audio playback of sounds, or playback via display of a graphical representation or a combination thereof.

A plan view of the undersurface 104 of the monitoring apparatus 100 is shown in FIG. 4. Referring to FIG. 4, in this embodiment the muscle function sensor 106 includes a pair of electrodes 180 and 182 for sensing an electrical potential associated with functioning of the muscle. The electrodes 180 and 182 are disposed on the undersurface 104 of the flexible carrier strip 102. The electrode 180 includes a conductive area 184 for forming a low-impedance connection with the epidermis 122 of the human subject 120 shown in FIG. 2. A conductor 186 connects between the conductive area 184 and a through-connection 188 for carrying current to the sensor interface 150 of the microprocessor 112 on the outer surface 116 of the flexible carrier strip 102. Similarly the electrode 182 includes a conductive area 190, a conductor 192, and a through-connection 194 for carrying current to the sensor interface 150. For the embodiment including the electrodes 180 and 182 shown in FIG. 4, the sensor interface 150 would include electromyography circuitry for conditioning electrical potential signals generated by activation of the underlying muscles. Such circuitry may include impedance buffering and amplification circuits and may further include a rectification circuit for rectifying the muscle signals. The processor circuit 110 may be configured to further process resulting digitized signals produced by the ADC 140. Such processing may involve, for example, causing the microprocessor 112 to perform averaging, peak detection, Fourier analysis, correlation, and/or other common signal processing functions on the signal to extract physiological indications indicating activation of the muscle and/or indicating a force of activation of the muscle.

In one embodiment a conductive gel may be applied to the conductive areas 184 and 190 to facilitate the low-impedance electrical contact to the epidermis 122 for sensing of electrical potentials generated by muscle cells underlying the conductive areas. Adhesive may be applied to portions of the undersurface 104 other than the conductive areas 184 and 190 for adhering the flexible carrier strip 102 to the epidermis 122 of the subject.

In other embodiments the muscle function sensor 106 may be implemented using a pressure sensor or a strain gauge operable to produce signals representative of a pressure, strain, or forces associated with the functioning of the underlying muscle. For example, in one embodiment the muscle function sensor 106 may be implemented using one or more fiber optic strain sensors on the undersurface 104 of the flexible carrier strip 102.

A plan view of the outer surface 116 of the monitoring apparatus 100 is shown in FIG. 5. Referring to FIG. 5 in one embodiment the transducer 108 may be a microphone 200 for detecting mechanical stimuli in the form of sound waves. For the example of detecting Bruxism in a human subject, the microphone 200 produces signals representing sounds in the environment including sounds produced by the subject. In this embodiment the sensor interface 150 would include signal conditioning circuitry for amplifying received sound waves, which are then converted into a digital representation by the ADC 140. The microprocessor 112 is configured to further process the signals to determine whether signal characteristics correspond to indicia related to Bruxism. For example, the microprocessor 112 may perform a Fast-Fourier-Transform (FFT) on the signals received at the ADC 140 and may determine whether frequencies indicative of Bruxism are present in the stimulus signals. In one embodiment, the microphone 200 is calibrated to facilitate determination of a sound pressure level (SPL) of sound waves incident on the microphone for quantifying the severity of the Bruxism condition in the subject 120.

In an alternative embodiment, the transducer 108 may be implemented using a pair of microphone transducers including the microphone 200 and a second microphone 202. In the embodiment shown in FIG. 5, the microphones 200 and 202 are spaced apart along the flexible carrier strip 102 and the microprocessor 112 is configured to process the respective signals to detect a phase difference. The phase difference between the signals from the respective microphones 200 and 202 facilitates determination of an approximate direction to the source of the mechanical stimulus producing the sound waves. In one embodiment, the flexible carrier strip 102 may bear an orientation mark such as an arrow 204 for orienting the monitoring apparatus on the jaw area 124 of the human subject. When the microprocessor 112 detects that the received signals have phase characteristics indicating sound originating from a location other than the mouth region of the subject, the signals may be disregarded as noise or provided with a lower weighting than sound signals that have phase characteristics consistent with originating from the subject's mouth region.

In an alternative embodiment either of the microphones 200 may be replaced by a vibration transducer for detecting mechanical stimuli in the form of vibration waves. In the example of detecting Bruxism, vibrations due to the grinding of the subject's teeth may be processed in a similar manner to sound waves to identify vibration frequencies or other signal characteristics indicative of Bruxism.

Still referring to FIG. 5, in another embodiment the monitoring apparatus may include a motion transducer 206 for detecting mechanical stimuli in the form of movements of the living animal. The motion transducer 206 may be implemented using a commonly available accelerometer device that senses both orientation and movement and produces a digital output of movement data. In case of the human subject 120 shown in FIG. 3, signals produced by the motion transducer 206 may be used as an indication of the subject rolling over while sleeping and such movements may be correlated with onset of Bruxism, as detected by the muscle or stimulus signals described above.

Alternatively or additionally, the output produced by the motion transducer 206 may be used to provide an indication of an orientation of a portion of the living body to which the flexible carrier strip is adhered. For example, signals from the accelerometer may be used to indicate whether the human subject 120 is lying on his back, on one side, or on the other side, and the orientation may also be correlated with the onset of Bruxism.

Some accelerometers may be used for measuring low frequency vibrations, and in one embodiment the a single accelerometer based transducer 108 may be implemented in place of either the vibration sensor disclosed above or one of the microphones 200 or 202 shown in FIG. 5. The single accelerometer based transducer would thus be capable of detecting multiple mechanical stimuli, including vibration, movement, and orientation. In other embodiments where it is desired to extract higher frequency information, a vibration sensor having a wider frequency response may be selected to provide vibration signals having frequency components at higher frequencies.

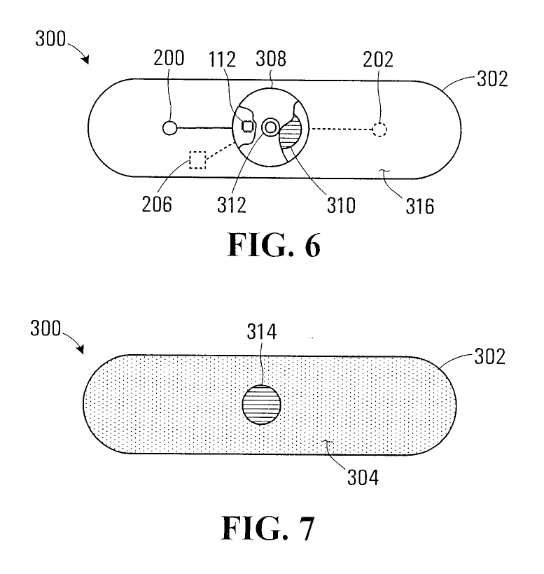

An alternative embodiment of a monitoring apparatus is shown in FIG. 6 and FIG. 7 generally at 300. Referring to FIG. 6, the monitoring apparatus 300 includes a flexible carrier strip 302 having an outer surface 316. A circuit housing 308 houses the processor circuit 110 generally as shown in FIG. 3 and also encloses an ultrasonic transducer 310. In the embodiment shown the circuit housing 308 includes a connector 312 for connecting an excitation signal to the ultrasonic transducer 310 for generating and delivering a dose of therapeutic ultrasonic radiation to the muscle underlying the flexible carrier strip 302. In this embodiment, the excitation signal is supplied by an external ultrasonic transducer driver since the transducer 310 would likely require power in excess of a power that can conveniently be delivered by the battery 130 (shown in FIG. 3). Referring to FIG. 7, in one embodiment a gel coupling area 314 is disposed on an undersurface 304 of the flexible carrier strip 302 directly below the ultrasonic transducer 310 and acts to provide a coupling medium for coupling ultrasonic radiation from the ultrasonic transducer to the underlying muscle.

The ultrasonic transducer 310 may also be in communication with the processor circuit 110, which may be configured to cause the dose of ultrasonic radiation to be initiated at the onset of Bruxism as detected by the monitoring apparatus disclosed above. The monitoring apparatus 300 may include any or all of the various sensors and transducers disclosed above in connection with the monitoring apparatus 100 for detecting various physiological indications.

Alternatively, the ultrasonic transducer 310 may be configured as a transceiver, which is operable to both generate ultrasonic radiation and to detect ultrasonic radiation reflected back to the transducer from the underlying muscle or other tissues. The processor circuit 110 may initially configure the ultrasonic transducer 310 as a generator for delivering an ultrasonic radiation pulse for coupling into the underlying muscle and tissue. The microprocessor 112 may then configure the ultrasonic transducer 310 to receive ultrasound radiation reflected from the underlying tissues. In this embodiment the processor circuit 112 may further be configured to provide physiological indications associated with functioning of the living animal based on changes in the reflected ultrasonic radiation over time.

As disclosed above, the monitoring apparatus 100 and monitoring apparatus 300 may be used in detecting and/or treating Bruxism. In other embodiments, the monitoring apparatus 100 and 300 may be used in producing physiological indications associated with other sleep disorders, such as snoring and sleep apnea, for example.

As disclosed above, the physiological indications stored in the flash memory 149 of the microprocessor 112 may be downloaded to the external host system 160 via the communications interface 146. In one embodiment the processor circuit 110 may be configured to process the stored data to produce an abridged version of the received periodic mechanical stimuli before downloading to the external host system 160. The abridged version may be generated by correlating portions of the stimulus signal with muscle activation provided by the muscle signal. The external host system 160 may be configured to provide playback of the abridged version of the mechanical stimuli to the subject 120.

The above disclosed embodiments of the monitoring apparatus provide for convenient attachment to a living animal, and may be configured as described to provide a range of physiological conditions that are useful in monitoring various disorders.

While specific embodiments of the invention have been described and illustrated, such embodiments should be considered illustrative of the invention only and not as limiting the invention as construed in accordance with the accompanying claims.

What is claimed is:

1. An apparatus for monitoring a living animal, the apparatus comprising:
a flexible carrier strip having an undersurface for adhering to an epidermis of the living animal;
a muscle function sensor disposed on the flexible carrier strip and operable to generate a muscle signal indicative of functioning of a muscle underlying the flexible carrier strip;
a transducer disposed on the flexible carrier strip operable to generate a stimulus signal in response to receiving mechanical stimuli;
a processor circuit disposed on the flexible carrier strip, the processor circuit comprising:
a sensor interface in communication with the muscle function sensor and the transducer for receiving the muscle and stimulus signals;
a microprocessor operably configured to process the signals to produce a physiological indication associated with functioning of the living animal;
a memory operable to store data representative of variations in the physiological indications over a period of time; and
a communications interface for communicating stored data to an output device.

2. The apparatus of claim 1 wherein the muscle function sensor comprises a pair of electrodes for sensing an electrical potential associated with functioning of the muscle, the electrodes being disposed on the undersurface of the flexible carrier strip.

3. The apparatus of claim 1 wherein the muscle function sensor comprises at least one of:
a force sensor disposed to sense a clamping force associated with activation of the muscle; and
a strain gauge disposed to sense strain in the epidermis underlying the flexible carrier strip.

4. The apparatus of claim 1 wherein the muscle function sensor is operably configured to produce a muscle signal indicating a force associated with the functioning of the muscle.

5. The apparatus of claim 1 wherein the transducer comprises a microphone and wherein the mechanical stimuli comprise sound waves.

6. The apparatus of claim 5 wherein the microphone is operable to produce a stimulus signal that facilitates determination of a sound pressure level of sound waves incident on the microphone.

7. The apparatus of claim 1 wherein the transducer comprises a vibration transducer and wherein the mechanical stimuli comprise vibration waves.

8. The apparatus of claim 1 wherein the transducer comprises a motion detector and wherein the mechanical stimuli comprise movements of the living animal.

9. The apparatus of claim 8 wherein the stimulus signal is operable to provide an indication of an orientation of a portion of the living body to which the flexible carrier strip is adhered.

10. The apparatus of claim 1 wherein the sensor interface comprises a signal conditioner for receiving the muscle and stimulus signals and converting the signals into a form suitable for processing by the microprocessor.

11. The apparatus of claim 1 wherein the flexible carrier strip is configured to be adhered to the epidermis of a human for producing physiological indications associated with sleep disorders.

12. The apparatus of claim 11 wherein the flexible carrier strip is configured to be adhered to the epidermis in one of a jaw area and a facial area.

13. The apparatus of claim 11 wherein the physiological indications associated with sleep disorders comprise at least one of:
clenching of jaw muscles associated with bruxism;
snoring; and
sleep apnea.

14. The apparatus of claim 1 wherein the communications interface is operable to generate signals for communication with a playback device for playing back received periodic mechanical stimuli.

15. The apparatus of claim 14 wherein the processor circuit is operably configured to further process the stored data to produce an abridged version of the received periodic mechanical stimuli for playback.

16. The apparatus of claim 1 wherein the microprocessor is operably configured to process the signals by:
processing the muscle and stimulus signals to identify physiological events in each of the signals; and
identifying a time correspondence between physiological events in the respective signals.

17. The apparatus of claim 1 further comprising an ultrasonic transducer disposed on the flexible carrier strip and operable to receive an excitation signal for delivering a dose of therapeutic ultrasound radiation to the muscle underlying the flexible carrier strip.

18. The apparatus of claim 1 further comprising an ultrasonic transceiver disposed on the flexible carrier strip and wherein the processor circuit is operably configured to:
cause the ultrasonic transceiver generate a pulse of ultrasonic radiation for delivery to tissues of the living body underlying the flexible carrier strip;
cause the ultrasonic transceiver receive a signal representing reflections of the ultrasonic waveform from the tissues; and
process the signal received by the ultrasonic transducer to produce the physiological indication associated with functioning of the living animal.
